(19) United States
(12) Patent Application Publication
(10) Pub. No.: US 2010/0093066 A1
(43) Pub. Date: Apr. 15, 2010

(54) DECELLULARIZATION AND RECELLULARIZATION APPARATUSES AND SYSTEMS CONTAINING THE SAME

(75) Inventors: Doris A. Taylor, Saint Paul, MN (US); Stefan M. Kren, Minneapolis, MN (US); Matthew Jeffrey Robertson, Minneapolis, MN (US)

Correspondence Address: UNIVERSITY OF MINNESOTA;OFFICE OF THE GENERAL COUNSEL, 360 MCNAMARA, 200 OAK STREET SE, MINNEAPOLIS, MN, 55455, US

(73) Assignee: Regents of the University of Minnesota, Minneapolis, MN (US)

(21) Appl. No.: 12/547,021

(22) Filed: Aug. 25, 2009

Related U.S. Application Data
(63) Continuation-in-part of application No. 12/064,613, filed on Oct. 27, 2008.
(60) Provisional application No. 60/711,501, filed on Aug. 26, 2005.
(60) Provisional application No. 60/815,242, filed on Jun. 19, 2006.
(60) Provisional application No. 61/211,613, filed on Mar. 31, 2009.

(51) Int. Cl. C12M 3/00

(52) U.S. Cl. 435/284.1

(57) ABSTRACT

The invention described herein provides systems and apparatuses for an initial preparation of an organ or tissue scaffold comprising an extracellular matrix, and subsequent recellularization of the scaffold to ultimately form a resultant artificial organ or tissue incorporating the natural and original extracellular matrix. The techniques and equipment of the invention collectively minimize scaffold collapse, compression or physical damage to the organ as well as afford the advantages of significant maintenance of the initial natural structural and biochemical attributes of the organ or tissue. The invention is particularly useful in organ and tissue transplantation and repair.

### RELATED APPLICATION DATA

This application is a continuation-in-part of, and claims benefit under 35 U.S.C. §120 to, pending U.S. application Ser. No. 12/064,613 deposited with the U.S. Patent & Trademark Office on Feb. 22, 2008, which claims benefit under 35 U.S.C. §371 to International Application No. PCT/US2006/033415 filed Aug. 28, 2006, which claims benefit under 35 U.S.C: §119(e) to U.S. Provisional Application Nos. 60/711,501 filed Aug. 26, 2005 and 60/815,242 filed Jun. 19, 2006. This application also claims benefit under 35 U.S.C. §119(e) to U.S. Provisional Application No. 61/211,613 filed Mar. 31, 2009.

### BACKGROUND OF THE INVENTION

Biologically derived matrices have been developed for tissue engineering and regeneration and are known. Matrices and scaffolds developed to date, however, generally have numerous disadvantages associated with them. Currently used techniques and equipment for decellularizing organs or tissues can substantially damage the extracellular matrix and membranous tissues associated with a given organ or tissue both physically and biochemically. This in turn can compromise the quality and integrity of the scaffold or its constituents, thereby adversely affecting the use of the scaffold and successful recellularization of the scaffold. As a further disadvantage, current techniques and equipment can also compromise and attenuate desirable biochemical attributes that were present in the intact natural initial extracellular componentry of the organ or tissue. It is believed that preservation and maintenance of the natural original scaffold attributes—both in terms of structural, biological and biochemical integrity—is critical to enhancing the success of forming a recelled artificial organ or tissue from the decelled scaffold.

One currently known decellularization technique, for example, involves immersion of an organ or tissue into a chemical detergent composition to detach cellular material from an extracellular matrix. Such techniques can be used in conjunction with mechanical disruption to further effectuate removal of cellular debris from the organ or tissue. The disadvantage associated with these methods is significant compromise of the original intact scaffold in terms of both physical and chemical properties.

There exists a need in the organ and tissue preparation and transplant fields for decellularized and/or recellularized organs and tissue having improved structural, biological and biochemical quality of the extracellular matrix or scaffold.

### SUMMARY OF THE INVENTION

The invention provides systems and apparatuses for an initial preparation of an organ or tissue scaffold comprising an extracellular matrix, and subsequent recellularization of the scaffold to ultimately form a resultant (perfusable) artificial organ or tissue incorporating the natural and/or original intact structural and biochemical components, e.g., extracellular matrix and capsular materials. The invention is particularly useful in complex organ and tissue transplantation, reconstruction and repair.

The invention provides a decellularization apparatus comprising: a decellularization chamber; a decellularization composition reservoir; an organ/tissue ingress conduit connected to the decellularization composition reservoir for delivery of the composition into the organ or tissue, the ingress conduit comprising a natural anatomical conduit engagement structure (e.g., vascular or duct engagement structure); and a organ/tissue egress conduit comprising a natural anatomical conduit engagement structure (e.g., vascular engagement structure). The decellularization apparatus can further comprise an organ/tissue positioning structure. The decellularization apparatus is structured to 1) cooperate with and utilize the natural anatomical conduits (e.g., natural vasculature or duct) of the target organ or tissue for delivery of a decellularization composition throughout the organ or tissue; and 2) reduce and minimize residency of the decellularization composition associated with the organ or tissue. The decellularization apparatus preferably maintains an aseptic environment throughout the apparatus components.

The invention also provides a decellularization system comprising a decellularization apparatus as described above in combination with a decellularization composition. The combined features of the decellularization system effectuate detachment and separation of the cellular material from the target organ or tissue to produce the extracellular matrix-based scaffold without the need for, mechanical disruption techniques.

The invention further provides a recellularization apparatus comprising: an organ/tissue scaffold recellularization chamber; a reservoir; a pump; an oxygenator; a pressurizer; and cellular introduction system. The various components are interconnected through a plurality of fluid conduits transferring an organ/tissue maintenance solution throughout the apparatus and through the natural anatomical conduits (e.g., natural vasculature) of the organ/tissue scaffold and developing regenerated organ/tissue product. The apparatus comprises an organ/tissue fluid ingress conduit and egress conduit, both the fluid ingress conduit and fluid egress conduit being structured to cooperate with the natural anatomical conduits (e.g., natural vasculature) of the target organ or tissue. The ingress fluid conduit and egress fluid conduit can comprise an anatomical conduit (e.g., vascular) engagement structure. The apparatus can further comprise a one or more sampler/monitor sites on the fluid circuit. In one embodiment, more than one pressurizer can be included and positioned at varying locations relative to the recellularization chamber (e.g., pre-organ/tissue and post-organ/tissue pressurizer). Further additional components that can be used include a thermal controller, as well as an oxygenation solution sub-circuit that operates cooperatively with the oxygenator component.

The invention also provides a recellularization system comprising a recellularization apparatus as described above in combination with an organ/tissue scaffold maintenance solution (also referred to herein as the primary fluid) and regenerative cell medium.

The invention provides for an artificial organ or tissue reconstruction system comprising the decellularization apparatus in combination with the recellularization apparatus as described herein. Furthermore, the invention provides for an artificial organ or tissue reconstruction system comprising a decellularization apparatus with decellularization composition system, in combination with a recellularization apparatus together with a scaffold maintenance solution and regenerative cell medium.

In a further embodiment, the decellularization apparatus and system and the recellularization apparatus and system can be constructed to simultaneously accommodate a plurality of separate or individual organs or tissues of the same or different type. In yet another embodiment, the decellularization apparatus and system and the recellularization apparatus and system can be constructed to simultaneously accommodate a plurality of intact anatomically joined organs or tissues.

The above and other advantages will become apparent from the following disclosure.

### BRIEF DESCRIPTION OF THE DRAWINGS

The invention is further illustrated by the following drawings—none of which are intended to be construed as necessarily limiting the invention.

FIG. 1 is a general schematic diagram of the decellularization apparatus according to one embodiment of the invention.

FIG. 2 is an anatomical illustration of a portion of a human heart showing a portion of an apparatus and system in a cardiac decellularization arrangement according to one embodiment of the invention.

FIG. 3 is a general schematic diagram of a recellularization apparatus and system according to one embodiment of the invention.

FIG. 4 is an anatomical illustration of a portion of a human heart having a partial cut-away view of the aortic arch interior and a portion of the apparatus and system of a cardiac recellularization arrangement according to one embodiment of the invention.

FIG. 5 is an angled side view of an oxygenator component of the apparatus according to one embodiment of the invention.

FIG. 6 is an angled side view of a pressurizer component of the apparatus according to one embodiment of the invention.

FIG. 7 is an angled side view of a modified oxygenator component of the apparatus according to one embodiment of the invention.

FIGS. 8A and 8B collectively show SEM photographs of decellularized kidneys. FIG. 8A shows SEM photographs of a perfusion-decellularized kidney. FIG. 8B shows SEM photographs of an immersion-decellularized kidney.

### DETAILED DESCRIPTION OF THE INVENTION

As used herein, the term “comprising” means the elements recited, or their equivalent in structure or function, plus any other element(s) which are not recited. The terms “having” and “including” are also to be construed as open ended unless the context suggests otherwise. Terms such as “about,” “generally,” “substantially,” and the like are to be construed as modifying a term or value such that it is not an absolute, but does not read on the prior art. Such terms will be defined by the circumstances and the terms that they modify are understood by those of skill in the art. This includes at the very least the degree of expected experimental error, technique error, and instrument error for a given technique used to measure a value.

As used herein, the term “perfusion” within the context of the invention is meant to refer to a flow of fluid through an organ or tissue utilizing the anatomical conduits (e.g., natural vasculature) associated with the organ or tissue—in whole or in part. The term is intended to distinguish from “immersion,” which is contemplated as a method employing soaking or submersion of an organ or tissue. The term is also intended to distinguish from mere “rinsing” or washing, or other techniques, which involve substantial agitation and application of a liquid to the mere exterior surface of an organ or tissue. When used within the context of the instant invention, the term is meant to preclude substantial mechanical disruption techniques and techniques involving substantial mechanical or physical agitation as a primary means to remove debris.

As used herein, the phrase “organ or tissue” is meant to refer to the desired target organs or tissues to be decellularized and recellularized in conjunction with the techniques and apparatuses described herein. Organs and tissues contemplated by the invention include those which contain natural vasculature (or other natural anatomical conduits) capable of being employed as a delivery route for a solution to effectuate cell removal. Unless specifically mentioned otherwise, the term “organ” is intended to include partial organs, such as a single lobe of a liver.

As used herein, the phrase “natural anatomical conduit” is meant to refer to intact channels, portals, ducts, and/or vessels associated with the natural structure of a specific organ or tissue that can be utilized to deliver and transport the compositions used with the invention. Natural anatomical conduits that can be used include, but are not limited to, vasculature (e.g., arteries and veins) of organs and tissues, bile ducts and veins associated with the liver, ureter of the kidney, trachea and airway passages of the lung, ventricles of the brain (including lateral ventricles), esophagus of the stomach, and the like.

Solid organs generally have three main components: the extracellular matrix (ECM), cells embedded therein (which may include nerve cells if associated with the particular organ or tissue), and a vasculature bed. Certain solid organs can also include membranous components as well. The term “scaffold” as used herein is meant to refer to the remaining collective intact construct following removal of cells and cellular debris of the initial organ or tissue, which may or may not be subsequently recellularized.

Extracellular matrix (ECM) components include, but are not limited to, fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina.

One aspect of the invention is decellularization of a solid organ or tissue the invention to remove most or all of the cellular components while preserving the extracellular matrix (ECM) of the organ or the vasculature bed—both in physical/structural attribute as well as biochemical attribute. Subsequently, the decellularized organ or tissue, (i.e., the scaffold) can be employed for recellularization to create a biocompatible artificial organ or tissue. Successful decellularization can be defined as the absence of detectable perenchymal cells, myofilaments, endothelial cells, smooth muscle cells, and nuclei in histologic sections using standard histological techniques, e.g., staining procedures. Preferably but not necessarily, residual cell debris has also been removed from the cellularized organ or tissue.

For effective recellularization and generation of an organ or tissue, it is important that that morphology and the architecture of the ECM be maintained (i.e., remain substantially intact) during and following the process of decellularization. The term “morphology” is meant to refer to the overall shape of the organ or tissue or ECM, while “architecture” as used herein refers to the exterior surface, interior surface, and the ECM there between. The morphology and architecture are referred to collectively as the “structural integrity” of the initial starting natural organ or tissue. An important aspect of the apparatuses and systems of the invention is that they perform the decellularization and recellularization processes of the invention in a manner that substantially preserves and maintains both the “macro-architecture” and “micro-architecture” of the extracellular matrix both structurally and biochemically and, consequently, significantly enhances the overall quality of the resultant regenerated organ or tissue. This includes preservation of residential structures that accommodate cells in situ.

Solid organs and tissues that can be used in the invention include, but are not limited to, heart, liver, lung, gall bladder, skeletal muscle, brain, pancreas, spleen, kidney, uterus, and bladder, and portions thereof. A “substantially closed” natural anatomical conduit system (e.g., substantially closed vasculature system) with respect to an organ means that, upon perfusion with a liquid, the majority of the liquid is contained within the solid organ and does not leak out of the solid organ, assuming the major conduits, channels, portals, ducts, or vessels are cannulated, ligated, clamped, or otherwise restricted. Despite having a substantially closed natural anatomical conduit or substantially closed vasculature system, many of the solid organs listed above have a defined “entrance” and “exit” channel, conduits or vessels which are useful for introducing and moving liquid throughout the organ during perfusion. In addition to the solid organs described above, other types of organs or tissues such as, for example, trachea, ureter or spinal cord tissues that can be decellularized using the methods disclosed herein. Organs and associated natural anatomical conduit systems that can be used include, but are not limited to, vasculature (e.g., arteries and veins) of organs and tissues (e.g., heart), bile ducts and veins associated with the liver, ureter of the kidney, trachea of the lung, ventricles of the brain (including lateral ventricles), esophagus of the stomach, and the like.

A decellularized organ or tissue as described herein (e.g., heart or liver) or any portion thereof (e.g., aortic valve, a mitral valve, a pulmonary valve, a tricuspid valve, a pulmonary vein, a pulmonary artery, coronary vasculature, septum, a right atrium, a left atrium, a right ventricle, or a left ventricle, papillary muscle, SA node, or liver lobe), with or without recellularization, can be used for transplantation into a patient or further research and study. Alternatively, part or all of a recellularized organ or tissue as described herein can be used to examine, for example, cells undergoing differentiation and/or cellular organization of an organ or tissue. It is contemplated by the invention that the apparatuses and systems described herein can be employed to prepare partial or complete organ or tissue scaffolds and/or recellularized organs or tissues for sample preparation. Such samples can be further utilized in a variety of ways, e.g., histological studies, biological assays, reparative constructs, physiological or cellular research, and the like. The invention can be used to prepare scaffolds from organs or tissue presented in various conditions or states, e.g., scaffolds of any age, gender, species, natural or genetically engineered, post-injury/trauma conditions (e.g., infarct, stroke, heart failure, cirrhosis) and the like.

In general, the invention provides systems and apparatuses for an initial preparation of an organ or tissue scaffold comprising an extracellular matrix, and subsequent recellularization of the scaffold to ultimately form a resultant whole or partial artificial organ or tissue incorporating the natural and original extracellular matrix. The techniques and equipment of the invention collectively minimize scaffold collapse, compression or physical damage to the organ scaffold, as well as afford the advantages of significant maintenance of the initial natural structural, its architecture and geometry, and biochemical attributes of the organ or tissue. The invention is particularly useful in organ and tissue transplantation and repair.

The initial scaffold formation phase of the invention can be accomplished using a decellularization apparatus and system. The decellularization apparatus is structured to 1) cooperate with and utilize the natural anatomical conduits (e.g., natural vasculature or ducts) of the target organ or tissue for delivery of a decellularization composition throughout the organ or tissue; and 2) reduce and minimize residency of the decellularization composition associated with the organ or tissue. This phase of the invention also comprises a decellularization system comprising the decellularization apparatus in combination with a decellularization composition to effectuate detachment and separation of the cellular material from the target organ or tissue from the “inside out” to produce the extracellular matrix-based scaffold having minimal damage.

The organ and tissue reformation phase of the invention includes an apparatus structured to recellularize the scaffold prepared in the initial phase. The apparatus is constructed and configured to introduce a regenerative population of cells to the scaffold and incubate the recellularized scaffold to ultimately form a reconstituted or reformed organ or tissue formed from using the new cell population. This phase of the invention also includes a recellularization system comprising a recellularization apparatus in combination with a cellular medium.

In another aspect of the invention, the invention comprises the combination of: a decellularization apparatus structured to cooperate with the natural anatomical conduits (e.g., natural vasculature) of the target organ or tissue; together with a recellularization apparatus. The invention also includes the combination of the respective decellularization system comprising the decellularization apparatus and decellularization composition together with the recellularization apparatus and recellularization medium.

The apparatuses and systems included within the invention are constructed to cooperate and utilize the natural anatomical conduits (e.g., natural vasculature) of the target organ and tissue throughout the decellularization and recellularization phases of the organ or tissue preparation technique. As a result of employing the natural anatomical conduits (e.g., vasculature) within the process(es) in the absence of both immersion techniques and substantial mechanical disruption techniques, damage to both the structural and biochemical constitution of the extracellular matrix scaffold are significantly reduced and the natural integrity of the matrix of the scaffold can be substantially preserved. Accordingly, uniformity of delivery and distribution of the decellularization composition throughout organ occurs, as well as reduction and or avoidance of physical damage to the organ and its surface occurs. As a further advantage, the residency of the decellularization composition within and upon the target organ or tissue, as well as the cellular debris waste, is substantially minimized and reduced. The use of the natural vasculature and the rapid separation of debris and waste fluid from the organ or tissue significantly improve the quality of the scaffold, thereby significantly improving the quality of the reconstituted organ or tissue. The integrity of outer membranous materials is also substantially preserved. In sum, the structural and biochemical integrity of extracellular matrix is substantially maintained.

### Decellularization Apparatus and System

The invention provides a decellularization apparatus comprising: a decellularization chamber; a decellularization composition reservoir; an organ/tissue ingress conduit connected to the decellularization composition reservoir and structured to engage and delivery of a decellularization composition directly into the natural anatomical conduits (e.g., natural vasculature) of said target organ or tissue; and a organ/tissue egress conduit comprising a conduit (e.g., vascular) engagement structure. In a preferred embodiment, the ingress conduit comprises a natural anatomical conduit structure (e.g., vascular engagement structure). The decellularization chamber can further comprise an organ/tissue positioning structure. The decellularization apparatus is structured to 1) cooperate with and utilize the natural conduits (e.g., vasculature) of the target organ or tissue for delivery of a decellularization composition throughout the organ or tissue; and 2) reduce and minimize residency of the decellularization composition on the organ or tissue. A critical aspect of the decellularization apparatus and system of the invention is that decellularization of the organ/tissue and creation of the organ/tissue scaffold is achieved in the absence of both mechanical disruption and immersion techniques. The components of the decellularization apparatus collectively accommodate and cooperate with natural organ or tissue conduits and/or vasculature, e.g., arteries, arterioles, veins, ducts, channels, and the like. It is an important aspect of the invention that the natural conduits (e.g., vasculature) be utilized for delivery and effectuation of the decellularization process.

In a preferred embodiment, the decellularization apparatus is constructed to perform the process while maintaining an aseptic or sterile environment associated with the organ or tissue within the chamber. In one embodiment, the decellularization apparatus includes a decellularization chamber that can comprise a sealed chamber that is structured to position the target organ or tissue during decellularization so as to reduce and minimize residency time of excess decellularization composition and separate cellular debris from said organ or tissue.

Suitable vascular engagement structures for use with the decellularization apparatus of the invention include those which: a) mechanically interacts with the target organ or tissue are structured to engage and accommodate the natural anatomical conduits (e.g., natural vasculature) of the organ/tissue; b) maintains a contiguous fluid conduit between the delivery and/or efflux conduit(s) for transport of the decellularization composition into and/or out from the organ or tissue; c) is structured and dimensioned to cooperate with the particular dimensions associated with the selected conduit and/or vascular routes; and d) prevents or reduces exposure of exterior organ or tissue membrane to the decellularization composition throughout the process.

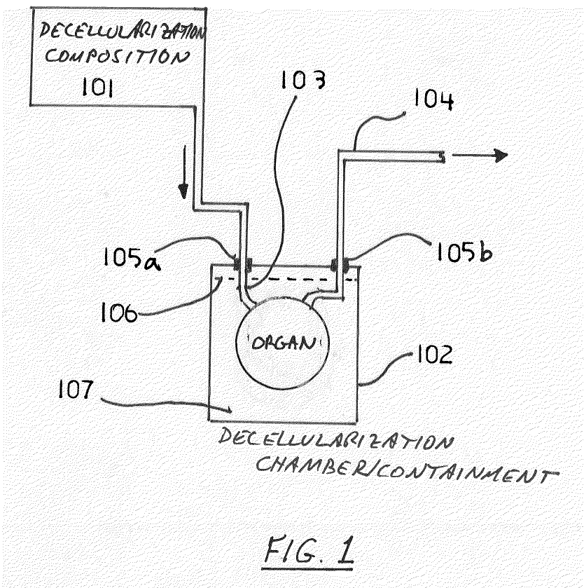

Referring now to FIG. 1, the decellularization composition can be initially stored within a decellularization composition reservoir 101 for subsequent delivery to the organ or tissue within the decellularization chamber 102. Direction of fluid flow in an operative system is represented by arrows in the diagram. The decellularization composition reservoir can be composed of any suitable material that can partake in sterile conditioning. The decellularization reservoir can be constructed with dimensions sufficient to contain the desired volume of decellularization composition within.

The decellularization composition reservoir 101 can comprise a fluid delivery conduit, i.e., the ingress conduit 103, to deliver the decellularization composition into the decellularization chamber 102 and directly into the input conduit or vasculature of the target organ or tissue contained within the decellularization chamber 102. The ingress conduit 103 can comprise a dispensation control mechanism (not shown), such as a valve, to regulate the amount and rate of decellularization composition flow into the target organ or tissue.

In an alternative embodiment (not shown), two or more chemically separated decellularization compositions, or two or more chemically separated ingredients in combination to create a decellularization composition, can be simultaneously or sequentially delivered. In this arrangement, two or more decellularization reservoirs can be employed which converge into a shared unitary fluid delivery conduit.

The components of the decellularization apparatus, e.g., fluid conduits and chambers, reservoirs, can be composed of any suitable material that can be sterilized or partake in sterile conditions and perform the function for that component. Suitable materials for the reservoir containment and decellularization chamber include, but are not limited to, glass and polymeric materials including plastics. Examples of suitable materials include medical grade glass, plastics and polymeric materials, metallic and metallic alloy materials. Materials that can be used can be rigid, semi-rigid or elastomeric, flexible and/or pliable. Suitable polymeric materials include, but are not limited to, polyethylene (PE), polytetrafluoroethylene (PTFE), PEEK, polyvinyl chloridine (PVC), silicone rubber, and the like. The various components of the apparatus can also be coated or treated to enhance their performance or afford properties as might be desired. The various components can be manufactured using conventional techniques and equipment readily available to those in the medical device field, such as thermoplastic molding techniques and equipment.

Residing within the decellularization chamber is the target organ(s) or tissue. The interior environment of the decellularization chamber is preferably sterilized and the chamber containment is preferably sealed. The entry point of the ingress conduit 103, and the exit point of the egress conduit 104, should be constructed so as to form an airtight seal associated with the relative juncture point(s) 105a and 105b respectively, into the decellularization chamber 102. In one embodiment, the juncture 105a of the ingress conduit 103 into the decellularization chamber 102 and the 105b juncture of the egress conduit 104 exiting the decellularization chamber 102 can comprise an elastomeric gasket seal to hermetically seal the exterior environment form the sterile interior environment.

Access into the decellularization chamber can be accomplished by various structures which permit both access and encasement of the contents within the chamber. A variety of suitable structures can be used provided they permit the formation of an airtight seal when closed. Examples include, but are not limited to, lids, hatches, and the like. In FIG. 1, a lid-type structure is represented by the presence of a dashed line 106 running horizontally across the upper region of the decellularization chamber.

The interior construction of the decellularization chamber can comprise an organ or tissue positioning structure (not shown). The positioning structure can vary in design, configuration and material according to the specific nature and attributes of the particular target organ or tissue to be decellularized. Preferably, the positioning structure is constructed to 1) closely replicate and mimic the natural anatomical orientation and suspension of the target organ or tissue during both the decellularization and recellularization processes; 2) accommodate the natural geometry and integrity of the intact or partial target organ or tissue; 3) reduce the likelihood of scaffold damage and collapse once decellularized; 4) reduce contamination/damage to scaffold; and 5) avoid biological or chemical incompatibility with subsequent recellularization, cell deposition and growth. In a preferred embodiment, the positioning structure further affords the ability to permit sterile maneuvering of the organ or tissue.

The positioning structure (not shown in the figures) can take a variety of forms and configurations according to the particular organ or tissue to be processed and provided the positioning structure can substantially participate in the prompt movement of the excess decellularization composition, fluid and cellular debris from and away from the target organ or tissue within the chamber. Examples of positioning structures that can be employed can include, but are not limited to, suspension elements, grates or screens, nets or mesh structures, semi-solid gels, and the like.

In one embodiment and for suspended organ and tissue arrangements, the interior dimensions and construction of the decellularization chamber can also comprise a gravitational fluid receptacle for the separation of and containment of excess fluids as may be dispensed from the organ or tissue before, during, and following the decellularization process. The fluid receptacle can vary in form and structure. The fluid receptacle can simply take the form of the chamber floor spaced apart from the contained organ or tissue (shown in FIG. 1 as 107). In another embodiment, the fluid receptacle can be in the form of a pocket or appendix to the primary portion of the decellularization chamber. A fluid receptacle affords the benefit of separating and rapidly removing the excess decellularization composition from the target organ or tissue.

In an alternative embodiment for more fragile and delicate organs and tissues, such organs and tissue can be used in association with pliable, semi-solid or viscous positioning structure, such as gels, sponges, and the like. Again as with the interior arrangements relative to the organ or tissue, it is preferred that irrespective of the positioning structure employed that the prolonged residency of the decellularization composition be avoided. This is an important aspect of the invention. Reducing or avoiding resident decellularization composition beyond what is necessary to remove cellular material from the scaffold enhances the preservation and maintenance of both the physical and biochemical integrity of the natural scaffold. This feature of the invention is responsible in part for the improved scaffold condition and integrity which, in turn, facilitates and enhances the subsequent recellularization stage.

In addition to the interior environment being sterile, the atmospheric conditions are also significant. Suitable temperature, pressure and humidity conditions to optimize preservation of the scaffold should be used. Temperatures for the chamber interior can be from about ambient temperature (22° C.) to about 40° C., preferably from about body temperature (37° C.) to about 40° C. Conventional and readily available equipment can be used to maintain environmental conditions within the decellularization chamber.

The egress conduit 104 permits transport and removal of excess decellularization composition as well as cellular debris from the organ or tissue and interior chamber environment. The egress conduit 104 can further comprise secondary or additional componentry as might be desired. For instance, the egress fluid pathway can include one or more output fluid sampling devices, measuring or monitoring devices, fluid movement components (i.e., pumps), and the like.

The terminal end of the vascular ingress conduit, and preferably the terminal end of the egress conduit as well, comprises a structure that engages and accommodates the natural anatomical conduit (e.g., natural vasculature) used for the delivery point of the decellularization composition into the organ or the tissue. Similarly, the initial entry point from the organ or tissue to the ingress conduit is also accomplished using a structure that engages and accommodates the natural conduits (e.g., vasculature) of the organ or tissue. Utilization of the natural anatomical conduits and/or vasculature of the target organ or tissue is a critical aspect of the invention. Accordingly, a critical feature of the conduit structures is that they are structured to interact with the natural native anatomy (conduits or vasculature) to deliver and remove the decellularization composition. Natural anatomical openings in the organ or tissue can serve as exit or outflow points for excess introduced fluids or mediums in the absence of an egress conduit.

At this decellularization stage of the process, however, this does not necessarily imply that the natural direction of blood flow correspond to fluid delivery direction of flow. It is important that the natural anatomical conduits (e.g., natural vasculature) be employed for decellularization, but this can entail, for example, 1) an arterial entry and venous exit pathway, 2) a venous entry and an arterial exit pathway of fluid flow, or 3) a ductal entry with vascular exit pathway. The particular conduit and/or vasculature arrangement to be employed can vary according to the particular organ or tissue.

For example, to decellularize a heart, a Langendorff perfusion arrangement can be used with ingress into the aorta and egress through the superior vena cava. To decellularize a liver, one can use the natural portal vein as the fluid entry route.

Again, the conduits of the decellularization apparatus of the invention can comprise a conduit (e.g., vascular) engagement structure (not shown) which a) mechanically interacts with the target organ or tissue are structured to engage and accommodate the natural conduits (e.g., vasculature) of the organ/tissue; b) maintains a contiguous fluid conduit between the delivery and/or efflux conduit(s) for transport of the decellularization composition into and/or out from the organ or tissue; c) is structured and dimensioned to cooperate with the particular dimensions associated with the selected conduit and vascular routes; and d) prevents or reduces exposure of exterior organ or tissue membrane to the decellularization composition throughout the process. The above criteria are important in order to preserve natural membrane material integrity of the intact organ or tissue, which is important to the improvement of scaffold quality and recelled organ quality as associated with the invention.

The conduit (e.g., vascular) engagement structure can take a variety of forms and materials. Such structures can comprise modified conduit terminal ends, such as reduced cross-sectional diameter or tapered diameter ends or end portions dimensioned for insertion into and within the receiving or discharging anatomical conduit or vessel. One conduit engagement structure can be in the form of a circumscribing clamp to secure the anatomical conduit or vessel over the terminal end of the conduit. Another embodiment can be in the form of an adapter, insert, segment or sleeve that couples to both the terminal end of the conduit and the anatomical conduit or vessel. Another embodiment of a conduit (e.g., vascular engagement structure) can be constructed as a flexible tubular extension of the conduit for insertion into the anatomical conduit or vessel.

In one embodiment, the conduit (e.g., vascular) engagement structure can comprise a conduit having a radially expanded end. This structure comprises an increased diameter at the juncture interfitting within the anatomical conduit or vasculature to create a fluid tight “seal.” As a further modification, adjacent the terminal end of the radially expanded end can be a circumscribing groove so as to facilitate placement of a clamp or tie and prevent or reduce the likelihood of slippage.

A wide variety of material(s) for the positioning structure and conduit (e.g., vasculature) engagement structure of the apparatus can be employed provided the material(s) are sterilizable and possess the desired structural integrity to perform the function within the apparatus. Examples of suitable materials include glass, plastics and polymeric materials, metals and metallic alloy materials. Materials that can be used can be rigid, semi-rigid or elastomeric, flexible and/or pliable. Suitable polymeric materials include, but are not limited to, polyethylene (PE), polytetrafluoroethylene (PTFE), polyvinyl chloridine (PVC), silicone rubber, and the like. The various components of the apparatus can also be coated or treated to enhance their performance or afford properties as might be desired.

In addition to engagement and cannulation of the conduits and vessels selected for delivery of the decellularization composition, other conduits and vessels not so employed can be ligated or clamped to control the containment and movement of the composition through the target organ or tissue. Ligation and clamping can be accomplished using conventional devices and techniques readily available to those skilled in the surgical arts.

Fluid flow, rate and pressure of the decellularization apparatus can be regulated and controlled passively by orienting the reservoir relative to the chamber in a manner permitting gravitational fluid flow. Alternatively, fluid flow and pressure can be regulated actively by one or more valves, pumps, or other control structures positioned at one or more points within the apparatus circuit. Flow rate, pressure, temperature and duration parameters can vary and be adjusted according to particular requirements and attributes associated with the specific organ or tissue. Pumps can be selected, controlled and/or positioned to provide variable or fixed rates, pulsatile or non-pulsatile flow, active ingress with passive egress, or passive ingress and active egress.

Alternating the direction of perfusion (e.g., anterograde or retrograde) can be employed to effectively and thoroughly decellularize the entire organ or tissue. Decellularization can be conducted at a suitable temperature and suitable duration appropriate for the target organ or tissue. Suitable decellularization temperatures can range from between about 4° C. and about 40° C. Suitable decellularization process duration can occur between a period from between about 2 hours and about 48 hours or longer, and including washing, can range from about 12 hours to about 96 hours or longer. Decellularization process time and temperature can be affected by numerous factors, such as age, size, condition and weight of the target organ and tissue, and supplemental techniques.

Decellularization of the Heart/Creation of Heart Scaffold

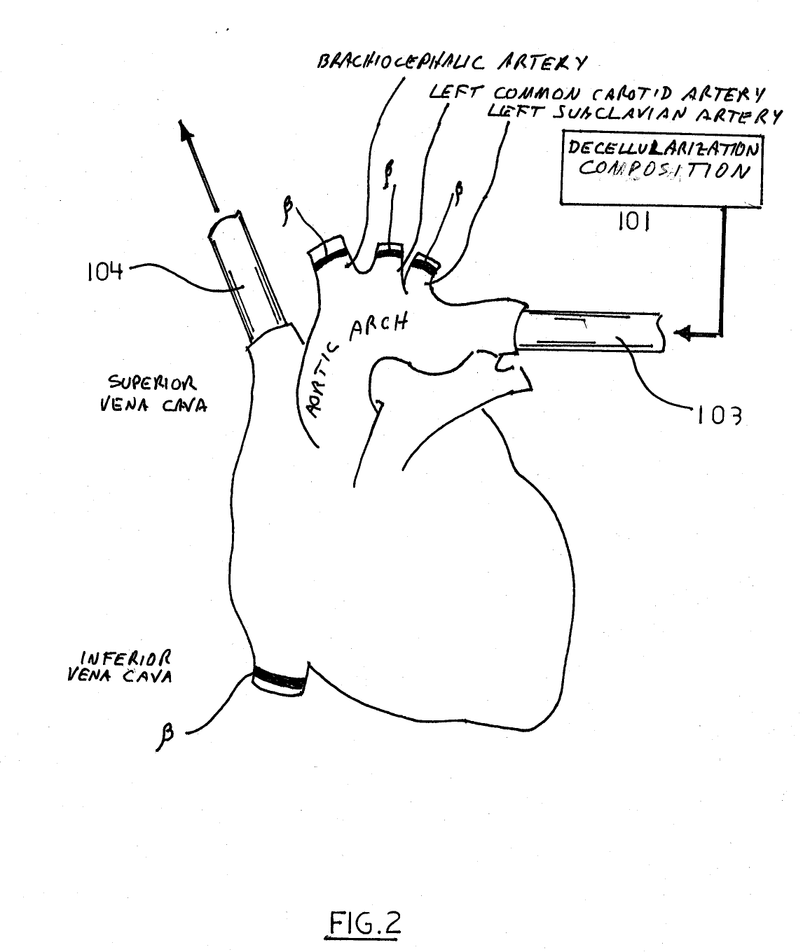

Referring now to FIG. 2 there is shown an illustration of one embodiment of an decellularization arrangement for the heart. The terminal portions of the vascular ingress conduit 103 and vascular egress conduit 104 are shown. For the heart, it is preferable to utilize the aorta for attachment of the fluid input. The remaining vessels (e.g., superior vena cava or output vessels) which are not employed for either influx or efflux of fluid from the heart can be clamped or ligated as represented by bands and the reference symbol (beta). In the figure, the vessels inferior vena cava, brachiocepahlic artery, left common carotid and left subclavian arteries are depicted as closed or ligated in FIG. 2.

The decellularization medium can be delivered into the heart via the aorta for perfusion throughout the organ utilizing the natural vasculature of the heart. The decellularization fluid can then exit the heart through the superior vena cava or other desired open (unclosed or non-ligated) exit locales of the heart. The vascular ingress conduit and vascular egress conduit and attachment componentry (cannulas and vasculature engagement structures) can be re-used and shared for the recellularization stage for the primary fluid or maintenance solution fluid circuit.

Decellularization Composition

A decellularization composition is used as part of a system in conjunction with the apparatus of the invention. The decellularization composition can be presented to the apparatus as a unitary composition to be delivered. Alternatively, the decellularization composition can include two or more chemically or temporarily separated ingredients for delivery through the apparatus. In this embodiment, the compositions can be delivered simultaneously or sequentially. For example, a first composition can comprise a detergent ingredient, and a second composition can comprise an enzyme. In any case, the invention contemplates that a decellularization composition comprising a cell disruption medium be included as part of the overall decellularization system part of the invention. The decellularization composition can be formulated according to the specific nature or attributes associated with the particular target organ or tissue.

Cellular disruptive ingredients that can be included within the decellularization composition can include, but are not limited to, detergents, surfactants, osmotic agents, chemical bases, enzymes, enzyme inhibitors, vaso-active chemical solutions, and combinations thereof.

Suitable detergents include but are not limited to SDS, PEG, Triton X, and combinations thereof. In certain embodiments, cell disruption media that can be employed as part of the decellularization composition can comprise an anionic detergent such as SDS and an ionic detergent such as Triton X or other surfactants.

Cellular disruptive ingredients that can be used also include water that is osmotically incompatible with the cells, and other osmotic agents. Suitable enzymes that can be used include, but are not limited to, collagenases, dispases, DNAses, and proteases, and combinations thereof. Enzyme inhibitors that can be used include, but are not limited to, protease inhibitors, nuclease inhibitors, collagenase inhibitors, and the like. Other chemical agents such as chemical bases can be used, including but not limited to, sodium hydroxide.

In addition to cell disruptive media, the decellularization composition can further comprise one or more secondary ingredients that are not “cell disruptive” by function and effect. Such secondary ingredients can include: nutritive agents, such as vitamins; therapeutic or pharmaceutical compounds and compositions; biologically active ingredients such as growth factors (VEGF, DKK-1, FGF, BMP-1, BMP-4, SDF-1, IGF and HGF), immune modulating agents (e.g., cytokines, glucocorticoids, IL2R antagonist, leucotriene antagonist) and/or factors modifying the coagulation cascade (aspirin, heparin-binding proteins, and heparin), hormones, and the like; and buffers such as PBS.

Alternatively, such ingredients can be perfused separately or subsequent to the decellularization step as part of the preparation and conditioning of the scaffold, e.g., extracellular matrix and vasculature bed. Furthermore, further treatments can be utilized as well after decellularization and before recellularization provided such treatments do not substantially adversely affect the desirable properties of the scaffold and are consistent with desired sterility of the process. An example of one such treatment can include irradiation (e.g., UV, gamma) so as to reduce or eliminate the presence of microorganisms remaining on or in a decellularized, organ or tissue. It may also be possible to effectuate hyper-cold solutions or “anti” freeze solutions while cooling to attenuate or eliminate microbes.

Recellularization Apparatus and System

The invention also includes an apparatus for recellularizing a decellularized organ extracellular matrix scaffold prepared as above, and a recellularization system including the recellularization apparatus, scaffold maintenance solution and regenerative cell medium. The recellularization apparatus is generally constructed so as to create and maintain both an internal fluid pathway within the organ or tissue scaffold utilizing the substantially intact vascular bed or anatomical pathways remaining after decellularization, and an external fluid environment relative to the organ, tissue or organ system. Put another way, the recellularization apparatus comprises a fluid circuit controlling both the exterior and interior fluid environment of the organ, tissue or system scaffold, and a fluid introduction system for delivery and deposit of a regenerative cell medium throughout the interior of the organ, tissue or system scaffold.

Overall, the sequential order of components of the recellularization apparatus can be as follows: reservoir, pump, oxygenator, pre-organ/tissue pressurizer, sampler/monitor site, organ/tissue recellularization chamber and cellular introduction system, and optional post-organ/tissue pressurizer and sampler/monitor. Further additional components that can be used include a thermal controller, as well as an oxygenation solution sub-circuit that operates cooperatively with the oxygenator component.

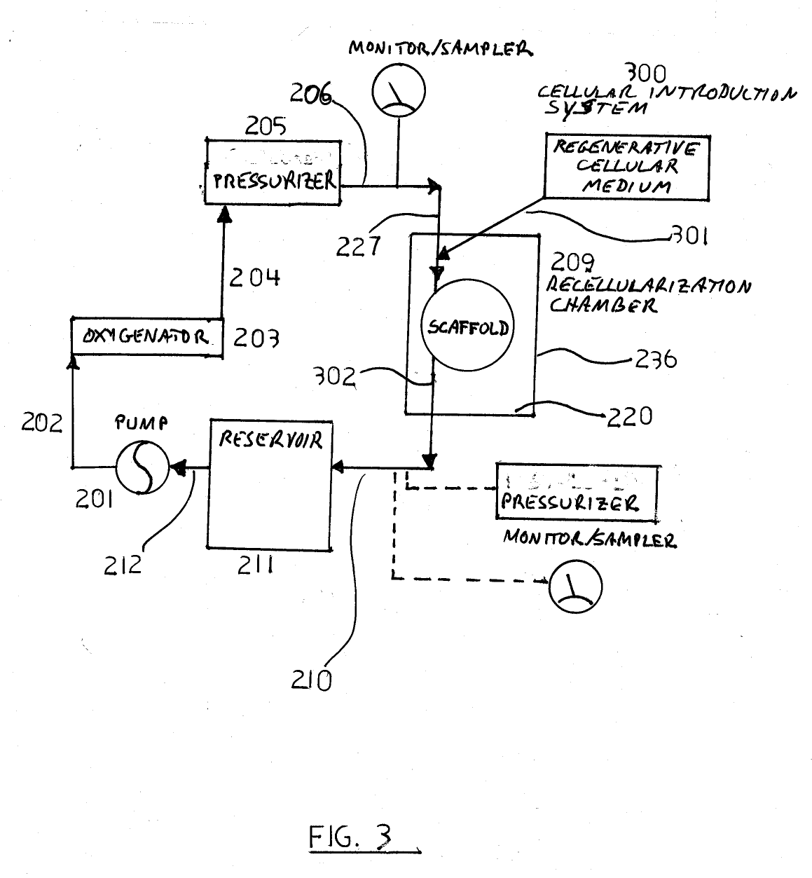

Referring now to FIG. 3, a schematic fluid circuit diagram of one embodiment of the recellularization apparatus and system is shown with fluid flow of an operative system represented by arrows in the diagram. The recellularization chamber 209 contains the organ or tissue scaffold within maintained by the maintenance solution (not shown) flowing through the fluid circuit and scaffold. Preferably, the primary fluid (maintenance solution) within the external fluid circuit is continually maintained at a temperature of about 37° C. to mimic body temperature as well as enhance cell viability of the regenerative cell medium when introduced into the scaffold.

The recellularization chamber 209 comprises a fluid containment 236 and is constructed with dimensions (e.g., height, width, length, depth) sufficient to accommodate the organ or tissue, or combination scaffold within. The recellularization chamber 209 can comprise an organ or tissue scaffold positioning structure (not shown). The scaffold positioning structure can vary in design, configuration and material according to the specific nature and attributes of the particular target organ, system or tissue to be recelled. The scaffold positioning structure can take a variety of forms and configurations according to the particular organ or tissue scaffold to be processed and provided the positioning structure can substantially participate in the prompt movement of the excess fluids, byproducts or metabolites from and away from the organ or tissue scaffold within the recellularization chamber.

Preferably, the scaffold positioning structure is constructed to 1) closely replicate and mimic the natural anatomical orientation and suspension of the target organ or tissue during both the recellularization process; 2) accommodate the natural geometry and integrity of the intact or partial target organ or tissue; 3) reduce the likelihood of scaffold damage and collapse; 4) reduce contamination/damage to scaffold; and 5) avoid biological or chemical incompatibility with recellularization cell deposition and growth. In a preferred embodiment, the positioning structure further affords the ability to permit sterile maneuvering of the organ or tissue scaffold. Examples of positioning structures that can be employed can include, but are not limited to, suspension elements, grates, shelves, or screens, nets or mesh structures, semi-solid gels, and the like. In one example, the liver can be supported using a glass shelf during decellularization to reduce liver deformation during the process.

In one embodiment and for suspended organ and tissue arrangements, the interior dimensions and construction of the recellularization chamber can also comprise a gravitational fluid receptacle 220 for the separation of and containment of excess fluids as may be dispensed from the organ or tissue during the recellularization process. The fluid receptacle can vary in form and structure. The fluid receptacle can simply take the form of the chamber floor spaced apart from the contained organ or tissue (as shown in FIG. 3, numerical reference 220). In another embodiment, the fluid receptacle can be in the form of a pocket or appendix to the primary portion of the recellularization chamber. In one embodiment, the fluid receptacle can include the entire recellularization chamber surrounding the tissue with an outlet.

In an alternative embodiment for more fragile and delicate scaffolds, such scaffolds can be used in association with pliable, semi-solid or viscous positioning structure, such as gels, sponges, and the like. It may be preferable that irrespective of the scaffold positioning structure employed, that the prolonged residency of the recellularization fluids is desirable to optimize recellularization. In the recellularization process, continual replenishment of nutrients is desirable and removal of metabolic waste or damaged cells is also desired. Certain organ and tissue types, such as cardiac tissues, are very oxygen consumptive so continual delivery of fresh media is important.

It may also be possible to include a filtration system as part of the fluid system (not shown). Sequential graded filters can be utilized in such a system. A filtration system can remove dead or damaged cells from the fluid in the system, as well as remove proteinaceous debris. In order to optimize recellularization results, it is preferable to remove damaged or dead cellular debris from the system.

The organ/tissue recellularization chamber 209 of the recellularization apparatus can comprise at least two conduits—an ingress conduit 227 and an egress conduit 302—relative to the recellularization chamber 209. The terms “ingress” and “egress” as used refer to the direction of the primary fluid/maintenance solution flow relative to entering and exiting the scaffold conduits, respectively. Together, fluid flow of the recellularization composition (maintenance solution combined with the regenerative cell medium) into the recellularization chamber 209 and throughout the organ/tissue scaffold is active flow that is transient in intra-organ/tissue residence, exiting out through the egress conduit 302 and onto the reservoir 211.

Recellularization composition exiting from the recellularization chamber 209 is then transported through conduit 210 into the reservoir 211 for transient residency and temporary storage. The recellularization composition (at this location of the circuit being maintenance solution and excess regenerative cellular medium) is drawn from the reservoir 211 by a fluid pump 201. Certain components are discussed in further detail as follows.

Oxygenator

A variety of oxygenation systems can be employed in the apparatus of the invention. In one embodiment, oxygenation can be accomplished by direct injection of carbogen gas into the maintenance fluid. This embodiment is less preferred, however, due to the generation of foam from the proteinaceous content in the fluid, which can lead to failure as pressure increases. Suitable oxygenators that can be employed should, therefore, preferably be reliable and provide sufficient oxygen to meet the biological demand of the organ or tissue materials being regenerated onto the scaffold.

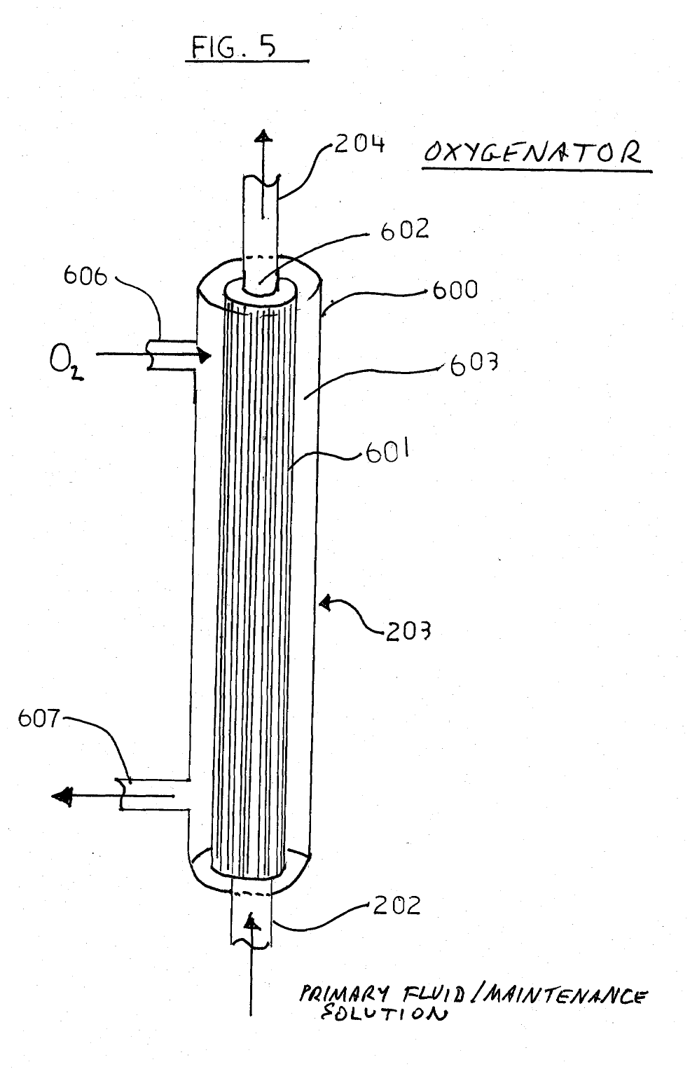

The apparatus of the invention can include an oxygenator 203. Suitable oxygenators that can be employed with the invention include those capable of introducing oxygen into a fluid in a dissolved state. Oxygenators termed “thin wall oxygenators” can be used, such as Media Sulfone™ D-150 Hemofilter oxygenator (available from Medica, Mendolla, Italy and illustrated in FIG. 5. This type of oxygenator device can include an elongated containment 600 having elongated fibrous film 601 separating two concentric fluid channels—an internal primary fluid channel 602 surrounded by a second oxygenating saline fluid channel 603 and partitioned from one another by the fibrous film 601. The primary fluid channel 602 is directly associated with the fluid transport of the apparatus can enter into the oxygenator via fluid conduit 202, run through the interior fluid channel 602 inside the fibrous film 601 (composed of gas permeable plastic tubing in film structure), and exit via fluid conduit 204. The oxygenating saline fluid channel 603 includes entry and exit ports (606 and 607, respectively) permitting flow of the oxygenating solution (not shown) alongside the fibrous film 601. In operation, oxygen transfers from the oxygenating solution passing through the fibrous film and permeates into the interior primary fluid in dissolved state.

Oxygenator Solution

The flow rate and chemical properties of the oxygenating solution can be coordinated with the established desired flow rate and tonicity conditions for the entire system. The formulation of the oxygenating solution can vary. Generally, the oxygenating solution cooperates with a primary fluid composition of having dissolved gas composition of about 5% CO₂ and about 95% O₂.

Tube Oxygenator Device

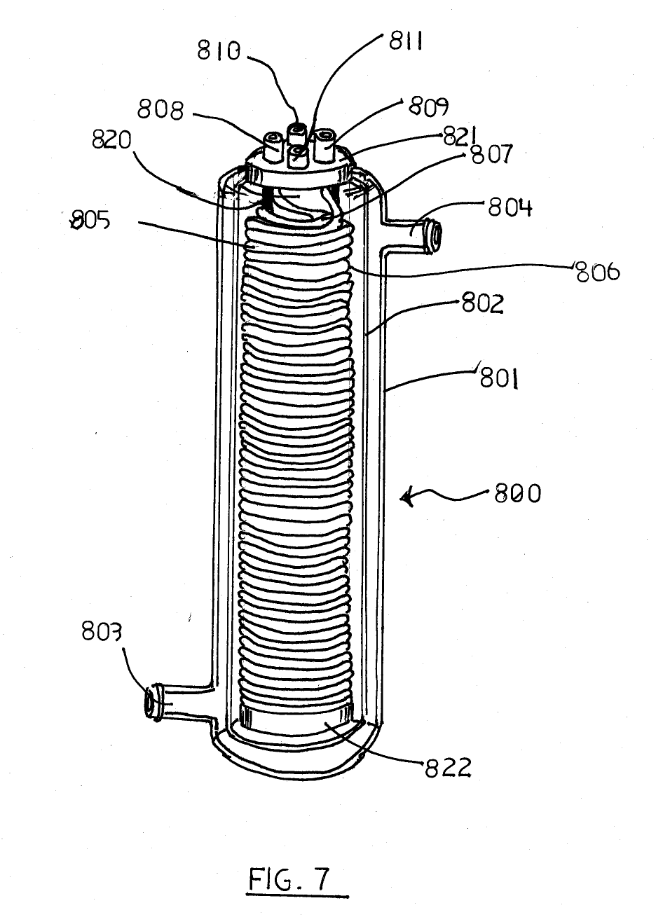

Alternatively and preferably, the apparatus of the invention employs a coiled tube oxygenator device as depicted in FIG. 7 for the oxygenator component of the apparatus. This oxygenator is structurally distinct from the above described oxygenator component in that the device is constructed to employ oxygen and carbon (carbogenic) gas directly around gas permeable tubing for passive diffusion into the maintenance solution and constructed for maintenance solution temperature control and maintenance as well.

Referring now to FIG. 7, the coiled tube oxygenator device 800 can comprise an outer containment 801 and inner containment 802, the inner containment 802 being concentrically positioned within the outer containment 801. The environment between the outer containment 801 and the inner containment 802 in the gap or space between the containments is physically separated from the internal environment within the inner containment 802. The outer containment 801 can be constructed to comprise an input port 803 and output port 804. Using a separate fluid transport and pump system (not shown), warmed water or fluid can be passed through the outer containment to effectuate temperature control of the maintenance solution passing through the oxygenator and facilitate control of the temperature for the entire system. Thus, the outer containment 801 and fluid passed through it can function as a “thermal jacket” for the inner components of the device 800.

The interior components of the oxygenator device 800 can be contained in part within the inner containment 802. The essential component for the interior structure comprises a gas permeable tubing 805 through which the maintenance solution passes. Maintenance solution can enter the tubing through fluid input port 808 and exit through fluid output port 809. Gas can be introduced into the inner containment 802 through gas input port 810 and exit through gas exit port 811. The fluid input port 808, fluid output port 809, gas input port 810 and gas exit port 811 can be located on the first endplate 821. First endplate 821 can be constructed to seal and contain the interior environment within the inner containment 802. Although the tubing 805 is depicted in coiled configuration and referred to as “coiled” herein, it will be understood that the tubing can be configured in a variety of other convoluted arrangements that place the tubing surface in intimate contact with carbogen gas and expose tubing surface to the extent sufficient to permit passive diffusion through the tubing material.

The tubing 805 can be composed of suitable gas permeable plastic or polymeric materials, such as silastic materials. In operation, gas introduced into the inner containment 802 passively diffuses through the tubing material and into the transported maintenance solution running through the tubing 805. As illustrated in the figure, the tubing 805 is shown in a double coiled arrangement with an outer coil portion 806 and inner coil portion 807 of the same contiguous tubing 805. The length of the tubing and cross-sectional diameter can vary. In one embodiment, about 50 feet of 0.078″×0.125″×50′ length tubing can be used.

The overall configuration and structural support for the tubing arrangement can be accomplished using support structures, which can be in the form of one or more elongated support rods 820 fixed to a first endplate 821 and second endplate 822 as illustrated. Support structure(s) used can take a variety of forms and configurations.

One advantage associated with the tube oxygenator device is the accomplishment of effective oxygenation of the maintenance solution without substantial or undesired agitation and foaming of the maintenance solution, which can denature the composition.

Pressurizer

The apparatus of the invention can comprise at least one pressurizer. The desirability of a bubble trap being included in the pressurizer will vary according to the design of the oxygenator. In the case of the tubing oxygenator described herein above, the necessity of a separate bubble trap is reduced or eliminated. The pressurizer can be active or passive, and can take a variety of forms, provided they can participate in a closed fluid circuit and permit viewing of the liquid passing through the device.

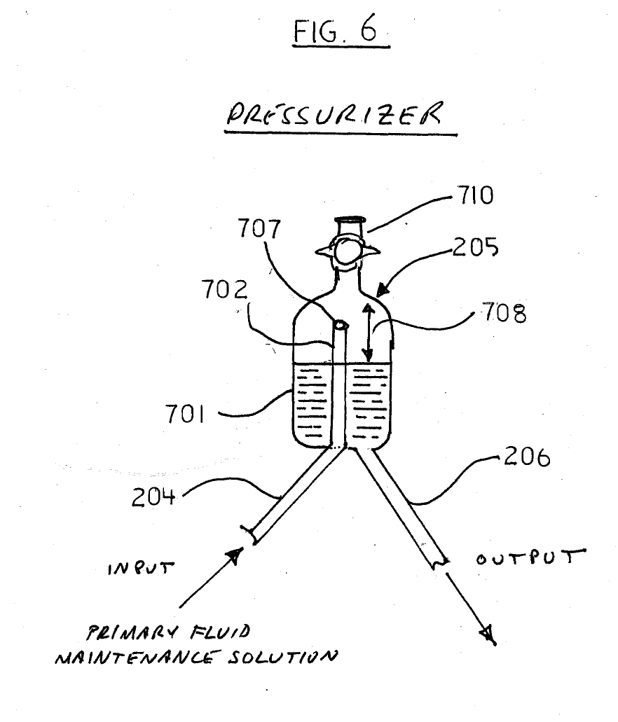

In one embodiment as illustrated in FIG. 6, the pressurizer 205 can be a passive device having a pressure chamber 701 having an elongated configuration and being vertically oriented and containing an open-ended standpipe 702 within which can be partially submerged in the primary fluid (shown as the liquid content) of the system in the pressure chamber 701. The device can comprise a sealable Luer lock access port 710 located at the upper-most portion of the pressure chamber 701 which can be sealed during operation of the entire apparatus. The pressure chamber 701 is positioned vertically such that during operation, a fluid level or meniscus is located in the medial region of the pressure chamber 701. The standpipe height is selected so that the upper portion and open end 707 of the standpipe 702 is raised above and higher than the fluid level/meniscus within the pressure chamber 701. In operation, primary fluid enters via fluid conduit (shown as fluid conduit 204 as part of the arrangement for the pressurizer) which itself becomes the standpipe 702. Fluid can exit the standpipe 702 at its open end 707 raised above the fluid level within the pressure chamber 701. The fluid (exiting) conduit 206 (in a pre-load pressurizer arrangement) of the pressure chamber 701 then transports primary fluid onward through the recellularization apparatus and system.

Manipulating the height of the fluid level and distance between the fluid meniscus and upper portion of the pressure chamber (distance being illustrated as 708) within the pressure chamber 701 controls the amount of fluid pressure of the fluid of the system (shown here as pre-load pressure) as positioned in the fluid circuit in advance of the organ/tissue chamber 209. The objective of the pre-load pressurization is to create an organ/tissue chamber completely filled with fluid medium with a top seal containment arrangement, as well as to provide in part biomimetic physiological pressure to the organ.

In one embodiment, the apparatus comprises a combination of both a pre-load pressurizer and post-organ/tissue pressurizer. This embodiment is preferred for cardiology (heart and cardiac tissues), i.e., that pre-load and post-load combination be utilized collectively in order to create or simulate natural in situ anatomical (pre-load and post-load) pressure conditions. Furthermore, the dual pressurizer arrangement reduces or eliminates reverse negative pressure against the organ/tissue chamber internal environment. Post-load pressurizer can be positioned at fluid conduit 210 exiting the organ/tissue recellularization chamber 209 as shown in FIG. 3.

Monitoring/Sampling

An important aspect of the apparatus and system of the invention is the precision of monitoring and controlling the combination of parameters and conditions—both physically and chemically. Monitored conditions include, but are not limited to, fluid pressure, flow rate, temperature, dissolved oxygen content, tonicity/saline, pH, metabolite concentration, and metabolism. It is important, therefore, that the apparatus design include sampling capabilities, such as a one or more monitoring and sampling sites. Monitoring and sampling site(s) can vary in number and location, depending on what is preferable for the most accurate measurements. Access sites or sensors contained enclosed within the system are possible, although no significant compromise of the sterility should occur with such.

Thus, the apparatus of the invention can comprise a sampling site 207. Sampling site can include one or more access ports to obtain samples, one or more monitoring electrodes (e.g., pH, O₂, pressure, tonicity, temperature, and the like), and combinations thereof. It is preferred that at least one sampling site be located on fluid conduit 208 immediately prior to organ/tissue chamber 209 so as to provide the most accurate measurements and indicia which would correspond to those within the contained organ/tissue chamber 209 environment surrounding the scaffold and ensure accuracy of the recellularization conditions.

Organ/Tissue Chamber

The organ/tissue chamber 209 component of the apparatus and system of the invention is the portion wherein the scaffold is contained and the recellularization process occurs. Preservation of an internal sterile environment within the organ/tissue chamber is critical to successful recellularization of the scaffold and subsequent implantation into a recipient. The overall dimensions of the organ/tissue chamber, i.e., length, width, height, volume, can vary according to a variety of factors. The selected dimensions must, however, as a minimum those which can internally accommodate the target organ, organ system, or tissue scaffold(s) within, as well as any structures and materials to aid in the positioning and orientation of the scaffold.

Both the intra-scaffold and extra-scaffold environment is controlled within the organ/tissue chamber of the apparatus and system of the invention. In operation, the scaffold is subjected to existent fluid flow controlled by the apparatus. Prior to placement of the scaffold within the organ/tissue chamber, the scaffold can be pre-treated or pre-coated with growth enhancing medium to facilitate attachment of the cells onto, or the growth of cells on, the extracellular matrix of the scaffold.

Cellular Introduction System

The apparatus of the invention further comprises a cellular introduction system 300 which introduces the regenerative cellular medium into the natural anatomical conduits (e.g., natural vasculature) of the organ or tissue. A cellular introduction system and components are generally illustrated in FIG. 3. The cellular introduction system can comprise one or more conduits specifically adapted for cooperation with, and utilization of, the intact scaffold conduit or vasculature bed and deliver regenerative (e.g., nutritive) cellular medium. The number and structure of the conduits and coupling arrangements associated with the cellular introduction system will vary according to the particular target organ or tissue. In general, the cellular introduction system 300 can comprise a cellular introduction catheter (generically represented as 301) which joins ingress conduit 227 to the scaffold, and which shares organ/tissue egress conduit 302. Typically, attachment and fixation of the cellular introduction system 300 will occur as part of the scaffold preparation and positioning inside the organ/tissue chamber. It is important that sterility be maintained throughout the preparation and positioning stage to the best extent possible.

For optimal recellularization results, there are a number of objectives that can affect recellularization and apparatus performance. First of all, it may be desirable to control partial or regional introduction of cells into the scaffold, if possible. Selective use of specific conduits e.g., vasculature, alone or in combination with site-specific injection of cells directly into the organ or tissue parenchyma can be employed alongside more remote general perfusion entry into major vessels. It is also preferable to replicate the natural anatomical orientation and suspension of the scaffold to mimic the natural in situ physical and chemical conditions of the corresponding intact organ or tissue. Third, it is important to reduce or prevent damage or collapse of the fragile, decellularized scaffold during recellularization. Ideally, excess cellular medium should be allowed to run off from the organ/system/tissue during recellularization being diluted into the circulating reservoir of medium.

Cellular Introduction System for Heart

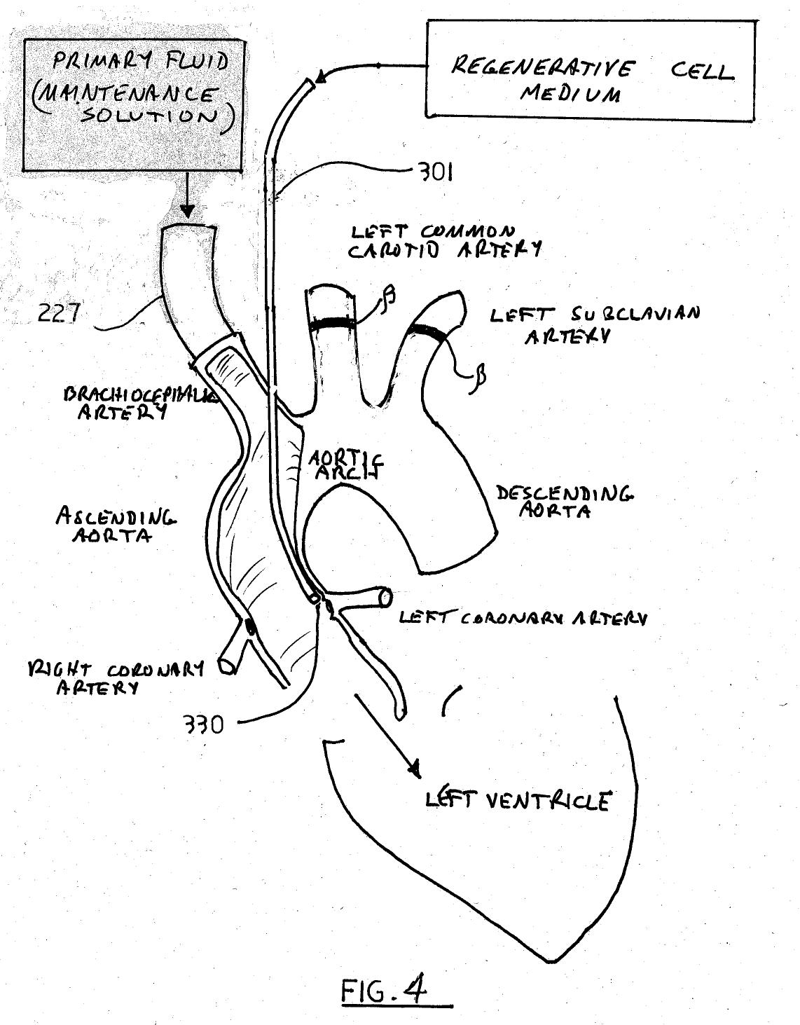

For a target scaffold where the heart is the organ, the following cellular introduction system can be employed and as shown in FIG. 4. For cellular introduction into the heart scaffold, the objective is to accomplish cellular flow as much into the coronary arteries as possible—preferably with simultaneous fluid flow within controlled by the apparatus. Variations are possible as well, such as parenchymal delivery of muscle cells onto the heart scaffold. Thus the positioning of the cellular introduction conduit(s) is important to achieve this objective. Another objective is to deliver the regenerative cell medium in the most uniformly distributing manner for the given target scaffold site as possible, which can be accomplished by delivery and deposit through the natural anatomical conduits alone, or in combination with (or supplemented by) site or localized injection of cells.

Referring now to FIG. 4, there is shown a diagram of a human heart and interior of the aortic arch with a cellular introduction catheter positioned therein. The distal open end 330 of the cellular introduction catheter 301 is shown positioned adjacent a coronary artery to i) deliver the regenerative cell medium in admixture with the active continuous flow of the primary fluid/maintenance solution delivered through the ingress conduit 227 facilitate, and ii) facilitate integral distribution of the regenerative cell medium throughout the natural vascular distribution of the heart (i.e., the global structure).

A cellular introduction catheter in the form of a polyethylene microcatheter can be used. Microcatheter dimensions can vary according to the species, e.g., for rodent cardiac scaffolds, the microcatheter can have a diameter of about 50 μm to about 100 μm. Larger microcatheter dimensions are possible for human cardiac scaffolds, for instance. The microcatheter can be introduced into the brachiocephalic artery of the aortic arch, which can have greater intact length throughout the decellularization and recellularization stages. The brachiocephalic artery permits comparatively lengthier vessel material for attaching and fixing a delivery cannula or catheter into the heart as a preferred influx/delivery site in the recellularization stage. Additionally, the brachiocephalic artery is positioned along the aortic arch such that there is optimal alignment for the catheter and subsequent delivery of the regenerative cell medium in close proximity to the coronary arteries during the recellularization stage which is a preferred arrangement.

The length of the microcatheter can be selected according to the natural geometry of the target heart scaffold such that the distal tip and open end of the microcatheter delivers the cells adjacent to and just above the aortic valve. Cellular delivery at this location facilitates entry of the cells into the coronary arteries to effectuate the desirable distribution throughout the cardiac scaffold vasculature. Following cellular delivery, the microcatheter can be removed and the created catheter opening in the brachiocephalic artery can be closed.

Various delivery arrangements for the regenerative cellular medium are possible. For instance, sequential introductions of different cell types over a short or long time period can be performed. Mixtures of different cell types can be simultaneously introduced or, alternatively, sequentially introduced over time as well. For instance, vascular cells may be preferred for vascular delivery.

Reservoir

Following the organ chamber, excess fluid and perfusate is transported via fluid conduit 210 and collected in reservoir 211. The dimensions (e.g., height and width, volume) can vary. In one embodiment, the reservoir can be in the form of a vertical column wherein the length of the reservoir is greater than the width.

The reservoir can operate in conjunction with additional equipment to optimize fluid conditions prior to recycling forward through the apparatus. In one embodiment, the reservoir can be combined with an oxygenator to replenish oxygen content into the fluid.

In an embodiment where the reservoir is absent an oxygenator, the volumetric capacity of the reservoir is a primary concern. In an embodiment wherein the reservoir is combined with an oxygenator, however, it is preferable that the reservoir be configured as a vertical column having a length greater than width. Vertically-oriented column structures for the reservoir/oxygenator combination facilitate dissolving of oxygen into the fluid while at the same time reducing the presence of bubble and emboli formation in the fluid.

In a further embodiment, temperature control systems can be combined with the reservoir. For example, the reservoir can be surrounded by a thermal water jacket to assist in maintaining the desired fluid temperature. Temperature control devices can be located on an apparatus component-by-component basis to maintain a desired temperature throughout the system. Alternatively, a temperature control device can be located in the incubator containment. This arrangement can, however, compromise access.

The reservoir can further comprise a fluid conduit 212 permitting and controlling recycling of the fluid as desired. It is important that to the extent possible, sterility of the fluid be maintained for recycled fluids re-entering the system circuit. Thus, it is possible to drain off or otherwise purge fluid at this juncture in order to control the extent of reintroduction and admixture of old fluid with fresh fluid which can be introduced at this point as well. Valve structures can be positioned at this location to permit these capabilities. Filters can also be positioned at this location as well, in order to permit sterile access.

In some embodiments it can be preferable to permit filling and removal of medium to and from the reservoir. This can depend upon the requirements or beneficial conditions for particular organs.

Pump

Fluid conduit 212 can be connected to the pump 201. The particular pump and pump action employed within the apparatus of the invention can be coordinated with the nature of the specific organ or tissue to be regenerated. In the case of recellularization of a heart scaffold, the mechanical training of the cells within the fluid medium is important. Accordingly, a pump constructed to participate in mechanical cell training of cardiac conditioning is preferred. A peristaltic pump can be employed in the apparatus of the invention, such as those available from Cole-Parmer MASTERFLEX™ (Cole Parmer, Vernon Hills, Ill.).

While peristaltic pumps can be used, preferably a pump which imitates or mimics a fast attack wave form with subsequent gentle release is used for an apparatus for heart scaffold recellularization. Suitable pump types that can be used for heart recellularization include rhythmic pump devices effectuating cardiac pump wave patterns. Such pumps can be piston-based devices such as Hugo Sacks Series 1400 (Hugo Sachs, Hugstetten, Germany) which independently control both volume and rate and, thus, can mimic natural heart pump effect and a cardiac wave form. Fluid transported away from the pump 201 toward subsequent components in the apparatus can be conducted through fluid conduit 202.

As with the decellularization apparatus, the components of the recellularization apparatus, e.g., fluid conduits and chambers, reservoirs, can be composed of any suitable material that can be sterilized or partake in sterile conditions and perform the function for that component. Suitable materials for the reservoir containment and decellularization chamber include, but are not limited to, glass and polymeric materials including plastics. Examples of suitable materials include medical grade glass, plastics and polymeric materials, metals and metallic alloy materials. Materials that can be sued can be rigid, semi-rigid or elastomeric, flexible and/or pliable. Suitable polymeric materials include, but are not limited to, polyethylene (PE), polytetrafluoroethylene (PTFE), polyvinyl chloridine (PVC), silicone rubber, and the like. The various components of the apparatus can also be coated or treated to enhance their performance or afford properties as might be desired. The various components can be manufactured using conventional techniques and equipment readily available to those in the medical device field, such as thermoplastic molding techniques and equipment.

The invention includes a recellularization system comprising the recellularization apparatus described herein in combination with a regenerative cell medium for reconstituting the organ or tissue scaffold. The invention further includes a recellularization system comprising the recellularization apparatus described herein; a regenerative cell medium for reconstituting the organ or tissue scaffold; and organ or tissue scaffolds.

An important aspect of the recellularization apparatus, selected operational parameters and compositions, and the recellularization system collectively, is the achievement and maintenance of biomimetic conditions relative to the natural organ or tissue in a living system. For this reason, it is preferable that the apparatus include access ports for physiological inspection and monitoring the apparatus operation and the system and the various process and compositional parameters, conditions, attributes, characteristics, properties, cell viability, and function to increase the chances of successful recellularization of the scaffold and create the artificial organ or tissue. These factors will vary according to the particular organ or tissue to be reconstructed.

Recellularization Composition

The recellularization process is performed using a combination of a recellularization composition. The recellularization composition can comprise two general compositions within: 1) a maintenance solution; and 2) regenerative cellular medium.

Scaffold and Organ/Tissue Maintenance Solution

The maintenance solution is formulated to preserve and maintain the physiological and chemical conditions of the contextual natural fluids surrounding the organ or tissue in vivo. These physiological and chemical conditions include, for example, salt concentration/tonicity, pH, nutritive, buffers, oxygen/carbon dioxide balance. In addition to the chemical and biochemical environment of the organ or tissue scaffold, the maintenance solution is preferably compatible with the recellularization (regenerative cellular) medium. Put another way, the maintenance solution can be formulated to maintain the scaffold and facilitate cellular regeneration of the scaffold as it develops into the regenerated organ, system or tissue. The maintenance solution ingredients and amounts are organ/tissue specific and will vary according to the specific organ or tissue involved. Furthermore, maintenance solution formulation can be modified over the recellularization process to optimize results at different stages of scaffold cellularization and differentiated or organized cellular development and integration into the scaffold.

Suitable mammal-derived serum can be obtained from a variety of mammalian sources. Examples of mammal-derived serum that can be used include, but are not limited to, fetal bovine calf serum (FBS), horse serum, and combinations thereof. It is also possible to use serum-free compositions. Suitable antimicrobials that can be used include, but are not limited to, penicillin, streptomycin, and amphotericin B.

One example of a suitable maintenance solution for use in heart scaffold recellularization comprises a combination of Iscove's Modified Dulbecco's Medium (IMDM)(1×) liquid (available from Invitrogen Corp., Carlsbad, Calif.) together with the additional ingredients: fetal bovine serum, penicillin/streptomycin, 1-glutamine, β-mercaptoethanol, horse serum, calcium chloride, magnesium chloride, and ascorbic acid (vitamin C). Iscove's Medium is generally composed of a mixture containing amino acids, vitamins, inorganic salts, as well as additional secondary ingredients. One example of prepared maintenance solution comprising Iscove's Medium and additional ingredients is set forth in the following table:

TABLE 1
Maintenance Solution - Heart and Cardiac Tissues

| | | | Amount |
|---|---|---|---|
| Ingredient | Amount | | (%) |
| Iscove's Medium* | 428.09 | mL | 85.618 |
| Fetal bovine calf serum 10% | 50.0 | mL | 10 |
| Antibiotic** | 5.0 | mL | 1 |
| L-glutamine | 5.0 | mL | 1 |
| β-mercaptoethanol | 910 | μl | 0.182 |
| Horse serum | 10 | mL | 2.0 |
| Salt solution*** | 1 | mL | 0.2 |
| Sodium heparin (50 Ku) | 45 | mg | 0.00009 |
| Total | 500.00 | mL | 100.00 |

*Iscove's MD Medium (available from Invitrogen Corp., Carlsbad, California).
**Antibiotic can be penicillin/streptomycin.
***Salt solution = 813.2 mg MgCl*6H₂O and 665.9 mg CaCl₂ and 250.0 mg ascorbic acid in dH₂O.

Regenerative Cellular Medium

The recellularization system of the invention in addition to the recellularization apparatus described herein, further comprises a regenerative cell medium. The reconstituted artificial organ or tissue can be generated by contacting a decellularized organ or tissue scaffold as described herein with a population of regenerative cells.

Regenerative cells as used herein are any cells used to recellularize a decellularized organ or tissue. Regenerative cells can be totipotent cells, pluripotent cells, multipotent cells, mature or immature cells, and can be uncommitted or committed. Regenerative cells also can be single-lineage cells alone or in combination. In addition, regenerative cells can be undifferentiated cells, partially differentiated cells, or fully differentiated cells. Regenerative cells as used herein include embryonic stem cells (as defined by the National Institute of Health (NIH); see, for example, the Glossary at stemcells.nih.gov on the World Wide Web). Regenerative cells also include progenitor cells, precursor cells, and “adult” derived stem cells including umbilical cord cells and fetal stem cells. Regenerative cells further include adult organ cells of non-stem or progenitor cells types, such as vascular and parenchymal cells, and inducible pluripotent stem cells. In one embodiment, combinations of different cells, or cell “cocktails” containing different cell population types, can be employed to reconstruct the target organ or tissue.

Examples of regenerative cells that can be used to recellularize an organ or tissue include, without limitation, embryonic stem cells, inducible pluripotent stem cells, umbilical cord blood cells, tissue-derived stem or progenitor cells, bone marrow-derived stem or progenitor cells, blood-derived stem or progenitor cells, mesenchymal stem cells (MSC), skeletal muscle-derived cells, adipose-derived stem or progenitor cells, multipotent adult progenitor cells (MAPC), multipotent adult stem cells, amniotic fluid-derived cells, or urine-derived cells. Additional regenerative cells that can be used include cardiac stem cells (CSC), multipotent adult cardiac-derived stem cells, cardiac fibroblasts; cardiac microvasculature endothelial cells, or aortic endothelial cells. Bone marrow-derived stem cells such as bone marrow mononuclear cells (BM-MNC), stromal cells, endothelial or vascular stem or progenitor cells, and peripheral blood-derived stem cells such as endothelial progenitor cells (EPC) also can be used as regenerative cells. It may also be possible to use bone cells, bone marrow cells, neural and spinal cord cells, blood, fat, neural cells from tissue, liver, skin, heart, and the like. Any organ or tissue derived cells including primary cells may be possible to use.

The number of regenerative cells that is introduced into and onto a decellularized organ in order to generate an organ or tissue is dependent on both the organ (e.g., which organ, the size and weight of the organ) or tissue and the type and developmental stage of the regenerative cells. Different types of cells may have different tendencies as to the population density those cells will reach. Similarly, different organ or tissues may be cellularized at different densities. By way of example, a decellularized organ or tissue can be “seeded” with at least about 1,000 (e.g., at least 10,000; 100,000, 1,000,000, 10,000,000, or 100,000,000) regenerative cells; or can have from about 1,000 cells/mg tissue (wet weight, i.e., prior to decellularization) to about 10,000,000 cells/mg tissue (wet weight) attached thereto.

Regenerative cells can be introduced (“seeded”) into a decellularized organ or tissue by infusion or injection into one or more locations. In addition, more than one type of cell (i.e., a cocktail of cells) can be introduced into a decellularized organ or tissue. For example, a cocktail of cells can be injected at multiple positions in a decellularized organ or tissue or different cell types can be injected into different portions of a decellularized organ or tissue. Alternatively, or in addition to injection, regenerative cells or a cocktail of cells can be introduced by perfusion into a cannulated decellularized organ or tissue. For example, regenerative cells can be perfused into a decellularized organ using a perfusion medium, which can then be changed to an expansion and/or differentiation medium to induce growth and/or differentiation of the regenerative cells.

During recellularization, an organ or tissue is maintained under conditions in which at least some of the regenerative cells can reside, multiply and/or differentiate within and on the decellularized organ or tissue. Those conditions include, without limitation, the appropriate temperature and/or pressure, electrical and/or mechanical activity, force, the appropriate amount of O₂ and/or CO₂, an appropriate amount of humidity, and sterile or near-sterile conditions. During recellularization, the decellularized organ or tissue and the regenerative cells attached thereto are maintained in a suitable environment. For example, the regenerative cells may require a nutritional supplement (e.g., nutrients and/or a carbon source such as glucose), exogenous hormones or growth factors, and/or a particular pH.

Again, the recellularization apparatus, collective system and procedure preferably employ biomimetic conditions to enhance the likelihood of reconstructive success. In addition, cell growth and biomimetic conditions can be responsive to or require organ or tissue-specific physiological inputs. Physiological inputs can comprise flow rates, electrical or mechanical inputs, shear stress, and the like—collectively or separately. Biomimetic inputs can be advantageous to recapitulate the original organ either during developmental, post-natal, adolescent or adult conditions.

The organ or tissue environment within the recellularization chamber is important to the success of populating the scaffold with cells and optimally regenerating the organ or tissue. Preferably, the exterior surface of the organ or tissue is fluid “bathed” and continually coated with the maintenance solution and/or regenerative cell medium throughout the recellularization stage to enhance cell viability and the restructuring of the organ or tissue. The nature and extent of fluid conditions surrounding the organ or tissue can vary according to the specific nature of the organ or tissue. The fluid bathing of the organ or tissue can be intermittent or continuous, partial or complete. As the natural anatomical conduits of the organ or tissue are employed during the recellularization stage, mixtures of excess maintenance solution and regenerative cell medium can exit through natural conduits onto the organ or tissue surface and cover the exterior of the organ or tissue.

Regenerative cells can be allogeneic to a decellularized organ or tissue (e.g., a human decellularized organ or tissue seeded with human regenerative cells), or regenerative cells can be xenogeneic to a decellularized organ or tissue (e.g., a pig decellularized organ or tissue seeded with human regenerative cells). “Allogeneic” as used herein refers to cells obtained from the same species as that from which .the organ or tissue originated (e.g., related or unrelated individuals), while “xenogeneic” as used herein refers to cells obtained from a species different than that from which the organ or tissue originated.

In some instances, an organ or tissue generated by the methods described herein is to be transplanted into a patient. In those cases, the regenerative cells used to recellularize a decellularized organ or tissue can be obtained from the patient such that the regenerative cells are “autologous” to the patient. Regenerative cells from a patient can be obtained from, for example, blood, bone marrow, tissues, or organs at different stages of life (e.g., prenatally, neonatally or perinatally, during adolescence, or as an adult) using methods known in the art. Alternatively, regenerative cells used to recellularize a decellularized organ or tissue can be syngeneic (i.e., from an identical twin) to the patient, regenerative cells can be human lymphocyte antigen (HLA)-matched cells from, for example, a relative of the patient or an HLA-matched individual unrelated to the patient, or regenerative cells can be allogeneic to the patient from, for example, a non-HLA-matched donor.

Irrespective of the source of the regenerative cells (e.g., autologous or not), the decellularized solid organ can be autologous, allogeneic or xenogeneic to a patient. In certain instances, a decellularized organ may be recellularized with cells in vivo (e.g., after the organ or tissue has been transplanted into an individual). In vivo recellularization may be performed as described above (e.g., injection and/or perfusion) with, for example, any of the regenerative cells described herein. Alternatively or additionally, in vivo seeding of a decellularized organ or tissue with endogenous cells may occur naturally or be mediated by factors delivered to the recellularized tissue.

The progress of regenerative cells can be monitored during recellularization. For example, the number of cells on or in an organ or tissue can be evaluated by taking a biopsy at one or more time points during recellularization. In addition, the amount of differentiation that regenerative cells have undergone can be monitored by determining whether or not various markers or functions are present in a cell or a population of cells. Markers associated with different cells types and different stages of differentiation for those cell types are known in the art, and can be readily detected using antibodies, metabolic profiles, standard immunoassays, metabolic capabilities, physiological responses, etc. See, for example, Current Protocols in Immunology, 2005; Coligan et al., Eds., John Wiley & Sons, Chapters 3 and 11. Nucleic acid assays as well as morphological and/or histological evaluation can be used to monitor recellularization as can organ function.

In one embodiment, the decellularization and recellularization apparatuses and systems can be utilized in the preparation of medium to high throughput tissue preparations. Such tissue preparations can be used for research purposes, such as histological studies, toxicity studies, cell development studies, and drug development or drug testing studies. Such tissue preparations can be generated as tissue scaffold preparations at conclusion of the decellularization stage or, alternatively, as regenerated tissue preparations at the conclusion of both the decellularization and recellularization stages. Preferably and in one embodiment, the tissue preparations can be prepared in accordance with the dimensions compatible with standard 6-well, 12-well, 24-well or 96-well tissue culture dishes or assay plates that can be employed in existing laboratory assay equipment by those skilled in the art. Prepared well dishes or plates can be used by themselves, or in combination with flow systems for sampling the wells, and for keeping the systems alive and physiologically active.

The scaffolds and portions thereof, and partially or substantially recellularized organs or tissues and portions thereof, at various stages throughout the processes can be used in a variety of other applications are possible as well. For example, decellularized matrices can be used in stem cell biological studies. Partially or substantially recellularized matrices can be employed as life science tool, drug discovery and toxicity testing, “personalized” test beds, preparation of therapeutic pieces of an organ or tissue, and the like.

The invention includes an organ/tissue reconstruction system comprising the combination of both the decellularization and recellularization apparatuses and system in sequence. More specifically, the invention provides for an artificial organ or tissue reconstruction system comprising the decellularization apparatus in combination with the recellularization apparatus as described herein. Additionally, the invention provides for an artificial organ or tissue reconstruction system comprising a decellularization system in combination with a recellularization system as described herein. Accordingly, upon presentation of target organ or tissue for reconstruction, the decellularization apparatus and system can be employed to prepare the scaffold, which can then be transferred to the recellularization apparatus and system to create the final ultimate regenerated organ or tissue in accordance with the techniques and methodology of the invention.

The incorporation or addition of additional equipment and devices is contemplated by the invention provided such equipment and devices do not substantially interfere with the operation of the apparatuses and systems of the invention. For example, electrical stimulation leads (i.e., pacing leads) can be integrated into the recellularization apparatus and system when the target organ is the heart to re-initiate recellularized heart function.

In a further embodiment, the decellularization apparatus and system and/or the recellularization apparatus and system can be structured to simultaneously accommodate a plurality of separate or individual organs or tissues of the same or different type. In yet another embodiment, the decellularization apparatus and system and the recellularization apparatus and system can be structured to simultaneously accommodate a plurality of intact anatomically joined organs or tissues. In this arrangement, the apparatuses respective organ/tissue chambers and conduits can be dimensioned and constructed to perform the decellularization and recellularization techniques on a plurality (i.e., two or more) of the same organ or tissue at the same time, such as a pair of kidneys or heart vessel and lung combination. Alternatively, apparatuses respective organ/tissue chambers and conduits can be dimensioned and constructed to perform the decellularization and recellularization techniques on a plurality (i.e., two or more) of two different organs or tissues that are anatomically joined and remain intact to create the scaffold and recellularization/reconstructed artificial organ/tissue, such as a pair of lungs with vessels joined to the heart.

In both the decellularization apparatus and recellularization apparatus, the operation of the apparatus components can be partially or substantially automated or controlled by computer(s). Computer systems and laboratory software programs can be incorporated into the operation systems associated with each apparatus and its components. Accordingly, the apparatuses can be constructed to operate in conjunction with, and/or perform according to, pre-programmed process and/or condition parameters.

Various operative functions can be computer-controlled, e.g., conditions, parameters, and timing. As mentioned briefly herein above, the apparatus systems can possess the ability to monitor operative characteristics (e.g., pressure, volume, flow pattern, temperature, gases, pH, mechanical forces, electrical stimulation (e.g., pacing). Sensors can be used to monitor the apparatuses and provide feedback signals to automated or computerized control systems. For example, sonomicrometry, micromanometry, conductance measurements can be used to acquire pressure-volume or pre-load recruitable stroke work information relative to myocardial wall motion and performance for cardiac applications. Sensors can also be used to monitor the liquid pressure, temperatures of medium, scaffolds or organ/tissue, pH, flow rate, oxygen levels, biological activity, and the like. In addition to having sensors for monitoring conditions and parameters, means for maintaining or adjusting conditions and parameters can be included as well. Thermometers, thermostats, electrodes, pressure sensors, check and overflow valves, flow valves and the like can be incorporated into the apparatuses.

The invention is further illustrated by the following examples none of which are meant to be construed as necessarily limiting the invention to the particular details of the recited embodiment.

### EXAMPLE

### Example 1

### Comparative Data: Perfusion Organ Decellularization Versus Immersion Decellularization Techniques Using Kidney

Using a kidney as the organ, the organ was decellularized using the immersion methods described in U.S. Pat. Nos. 6,753,181 and 6,376,244, incorporated herein by reference. Briefly, an organ was placed in dH₂O and agitated with a magnetic stir bar rotating at 100 rpm for 48 hours at 4° C., and then the organ was transferred to an 10 ammonium hydroxide (0.05%) and Triton X-100 (0.5%) solution for 48 hours with continued magnetic stir bar (100 rpm) stirring of the solution. The solution was changed and the 48 hr immersion with the ammonium hydroxide and Triton X-100 was repeated as needed to decellularize the organ (generally a visual acellular organ). The liver took approximately 5 repetitions of ammonium 15 hydroxide and Triton X-100 to generate a visually acellular organ. After the decellularization process, organs were transferred to dH2O for 48 hours with agitation (again stirring at 100 rpm); lastly, a final wash was performed with PBS at 4° C. and stirring.

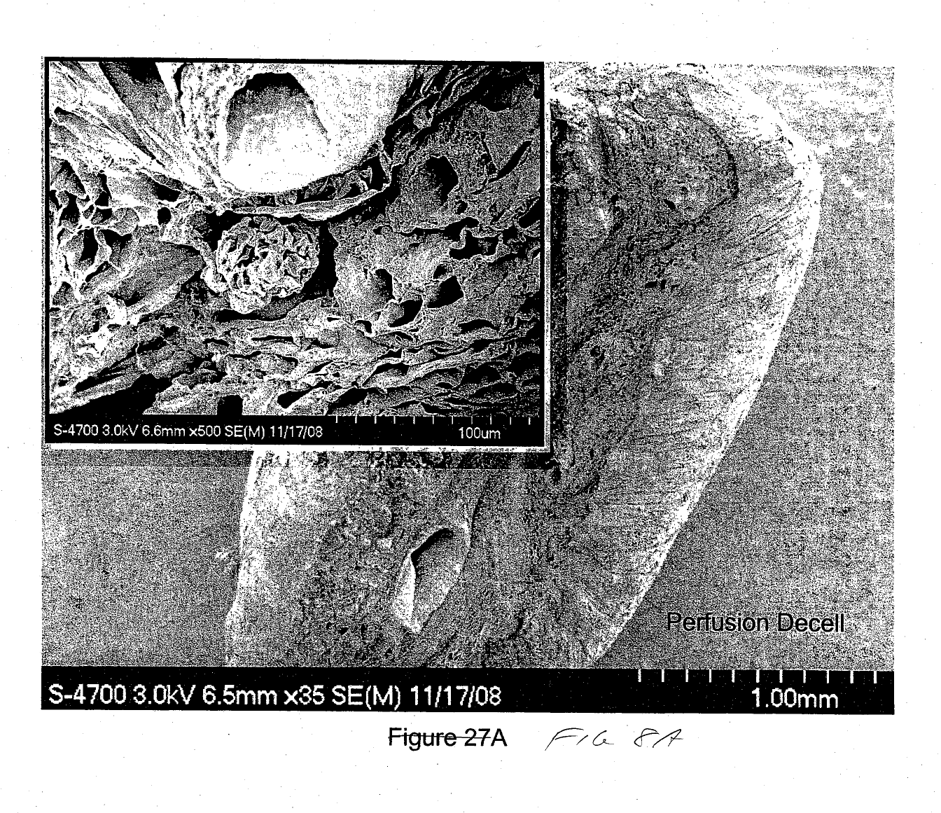

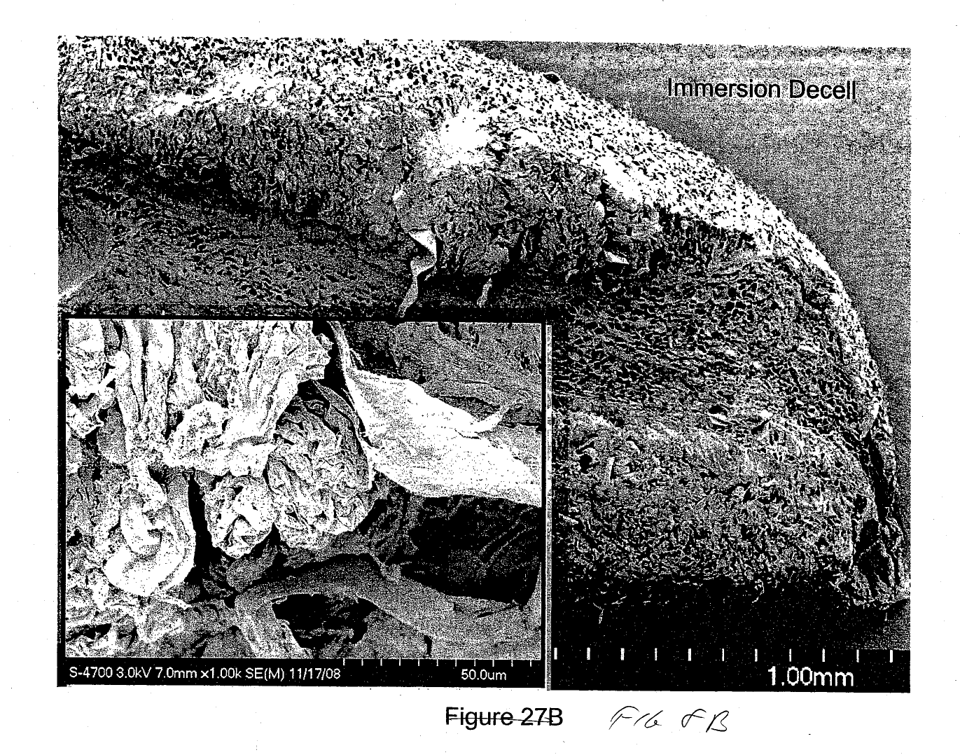

FIG. 8 shows SEM photographs of decellularized kidney. FIG. 8A shows a perfusion-decellularized kidney, while FIG. 8B shows an immersion decellularized kidney. These images further demonstrate the damage that immersion-decellularization caused to the ultrastructure of the organ, and the viability of the matrix following perfusion-decellularization.

### INDUSTRIAL APPLICABILITY

The invention is useful in the preparation of organ and tissue materials having enhanced quality and biocompatibility. The invention can be used to prepare artificial organs and tissues for transplantation and scientific research, as well as prepare various high quality organ and tissue samples for histological, toxicity, drug discovery or drug screening research purposes.

The invention herein above has been described with reference to various and specific embodiments and techniques. It will be understood by one of ordinary skill in the art, however, that reasonable variations and modifications may be made with respect to such embodiments and techniques without substantial departure from either the spirit or scope of the invention defined by the following claims. Where reference has been made to patents, applications and publications herein above, the full text of each are incorporated herein by reference.

What is claimed is:

1. A decellularization apparatus for removing cells from an organ or tissue, said apparatus comprising:
a decellularization chamber;
a decellularization composition reservoir;
an ingress conduit connected to the decellularization composition reservoir for delivery of said composition into said organ or tissue, said ingress conduit comprising a natural anatomical conduit engagement structure;
wherein said decellularization apparatus is structured to cooperate with and utilize the natural vasculature of said organ or tissue for delivery of said decellularization composition throughout the organ or tissue.

2. The decellularization apparatus according to claim 1, further comprising an egress conduit comprising a natural anatomical conduit engagement structure.

3. The decellularization apparatus according to claim 1, wherein said natural anatomical conduit engagement structure is a vasculature engagement structure.

4. The decellularization apparatus according to claim 2, wherein said natural anatomical conduit engagement structure is a vasculature engagement structure.

5. The apparatus according to claim 1, further comprising an organ/tissue positioning structure within said decellularization chamber.

6. A decellularization system comprising a decellularization apparatus as claimed in claim 1 in combination with a decellularization composition.

7. A recellularization apparatus comprising:
an organ/tissue scaffold recellularization chamber;
a reservoir;
a pump;
an oxygenator;
a pressurizer; and
a cellular introduction system;
wherein said components are interconnected through a plurality of fluid conduits.

8. The recellularization apparatus according to claim 7, wherein said fluid conduits comprise a fluid ingress conduit and fluid egress conduit being structured to cooperate with the natural vasculature of the target organ or tissue for delivery of fluid through the natural vasculature of a organ/tissue scaffold.

9. The recellularization apparatus according to claim 8, wherein said ingress fluid conduit and egress fluid conduit comprise a vascular engagement structure.

10. The recellularization apparatus according to claim 7, further comprising a sampler/monitor sites on the fluid circuit.

11. The recellularization apparatus according to claim 7, further comprising a pre-organ/tissue pressurizer and post-organ/tissue pressurizer.

12. The recellularization apparatus according to claim 7, further comprising a thermal controller.

13. The recellularization apparatus according to claim 7, further comprising an oxygenation solution sub-circuit that operates cooperatively with said oxygenator.

14. A recellularization system for regenerating an organ or tissue from a scaffold comprising: a recellularization apparatus as claimed in claim 7, in combination with an organ/tissue scaffold maintenance solution and regenerative cell medium.

15. The recellularization system according to claim 14, wherein said regenerative cell medium is delivered to said organ or tissue by said cellular introduction system.

16. An artificial organ or tissue reconstruction system comprising in combination, a decellularization apparatus and a recellularization apparatus, said decellularization apparatus comprising:
a decellularization chamber;
a decellularization composition reservoir;
a vascular ingress conduit connected to the decellularization composition reservoir for delivery of said composition into said organ or tissue, said vascular ingress conduit comprising a vascular engagement structure;
wherein said decellularization apparatus is structured to cooperate with and utilize the natural vasculature of said organ or tissue for delivery of said decellularization composition throughout the organ or tissue;
together with said recellularization apparatus comprising:
an organ/tissue scaffold recellularization chamber;
a reservoir;
a pump;
an oxygenator;
a pressurizer; and
a cellular introduction system;
wherein said components are interconnected through a plurality of fluid conduits.

15. An artificial organ or tissue reconstruction system according to claim 16, further comprising: a decellularization composition for use with the decellularization apparatus; and a scaffold maintenance solution and regenerative cell medium for use with the recellularization apparatus.

16. The decellularization apparatus according to claim 1 wherein said decellularization apparatus is structured to simultaneously accommodate a plurality of separate or individual organs or tissues of the same or different type.

17. The decellularization apparatus according to claim 16, wherein said apparatus is structured to simultaneously accommodate a plurality of intact anatomically joined organs or tissues.

18. The recellularization apparatus according to claim 7, wherein said recellularization apparatus is structured to simultaneously accommodate a plurality of separate or individual organs or tissues of the same or different type.

19. The recellularization apparatus according to claim 18, wherein said apparatus is structured to simultaneously accommodate a plurality of intact anatomically joined organs or tissues.
